(19) United States
(12) Patent Application Publication
(10) Pub. No.: US 2025/0032810 A1
(43) Pub. Date: Jan. 30, 2025

(54) NON-TRANSVENOUS IMPLANTABLE CARDIOVERTER DEFIBRILLATOR DEVICE FOR EMITTING A FIBRILLATION PULSE

(71) Applicant: BIOTRONIK SE & Co. KG, Berlin (DE)

(72) Inventors: Thomas DOERR, Berlin (DE); Ingo WEISS, Berlin (DE)

(73) Assignee: BIOTRONIK SE & Co. KG, Berlin (DE)

(21) Appl. No.: 18/715,531

(22) PCT Filed: Nov. 29, 2022
(86) PCT No.: PCT/EP2022/083646

§ 371 Date: May 31, 2024

(30) Foreign Application Priority Data
Dec. 14, 2021 (EP) 21214329.1

(51) Int. Cl. A61N 1/39

(52) U.S. Cl. CPC A61N 1/3956

(57) ABSTRACT

A non-transvenous implantable cardioverter defibrillator device comprises a generator device comprising a processing circuitry and a shock generation circuitry, and at least one lead comprising a shock electrode for emitting electrical shock pulses externally to a patient's heart. The processing circuitry is configured to identify a sensed ventricular contraction event in a sensed electrocardiogram signal or said processing circuitry is configured to control said shock generation circuitry to generate at least one conditioning pulse for emission by said shock electrode to cause an induced ventricular contraction event. The processing circuitry is further configured to control said shock generation circuitry to generate a fibrillation pulse for emission by said shock electrode at a delay time after said sensed or induced ventricular contraction event in order to induce a cardiac fibrillation state.

### CROSS-REFERENCE TO RELATED APPLICATIONS

This application is the United States National Phase under 35 U.S.C. § 371 of PCT International Patent Application No. PCT/EP2022/083646, filed on Nov. 29, 2022, which claims the benefit of European Patent Application No. 21214329.1, filed on Dec. 14, 2021, the disclosures of which are hereby incorporated by reference herein in their entireties.

### TECHNICAL FIELD

The instant invention concerns a non-transvenous implantable cardioverter defibrillator device and a method for operating a non-transvenous implantable cardioverter defibrillator device.

### BACKGROUND

A non-transvenous implantable cardioverter defibrillator device generally is designed for implantation external to a patient's heart. A non-transvenous implantable cardioverter defibrillator device, in short non-transvenous ICD, comprises a generator device having a processing circuitry and a shock generation circuitry, and at least one lead comprising a shock electrode for emitting an electrical shock pulse externally to a patient's heart. The lead is connected to the generator device. The generator device may, for example, be implanted subcutaneously in a patient. The lead, in a connected state, extends from the generator device, the lead being implanted such that it fully rests outside of the patient's heart. The lead may, for example, extend from the generator device towards a location in the region of the patient's sternum, the shock electrode hence being placed outside of the patient's heart for emitting an electrical shock pulse at a location external to the patient's heart.

The term “non-transvenous” in this respect in particular shall express that the lead of the non-transvenous implantable cardioverter defibrillator device does not extend transvenously into the heart, but fully rests outside of the patient's heart.

The non-transvenous implantable cardioverter defibrillator device in particular is designed for emitting electrical shocks in case life-threatening arrhythmias of a patient's heart are detected. By means of an electrical shock a defibrillation shall be achieved in order to reset the cardiac rhythm back to a normal state.

When implanting any cardioverter defibrillator device it is required to establish whether the cardioverter defibrillator device has been implanted such that it reliably may sense cardiac signals and may couple energy into the patient's heart in order to achieve a desired action. For this, typically a so-called threshold testing in the context of the implantation procedure is performed in order to evaluate whether a detected arrhythmia may be effectively ended by emission of a shock pulse using the cardioverter defibrillator device. For such a threshold testing, it is desirous to cause the patient's heart to enter into a fibrillation state. In the fibrillation state, a sensitivity of a sensing arrangement of the cardioverter defibrillator device may be tested, and the effectiveness of a coupling of the shock electrode may be assessed in order to potentially adapt the positioning of sensing electrodes as well as the shock electrode and to configure the setup of the cardioverter defibrillator device for its subsequent operation.

In some systems, sequences of pulse bursts are emitted by a shock electrode in order to cause a patient's heart to enter into a fibrillation state. As such pulse bursts may be emitted over a duration of several seconds and may be delivered with substantial energy, such pulse bursts may cause a significant stress on a patient and its muscular system, causing potentially postoperative pain to a patient.

There hence is a desire to enable a transitioning of a patient's heart into a fibrillation state for the purpose of performing a threshold testing with a minimum impact onto the patient.

In an apparatus and method disclosed in U.S. Publication No. 2015/0306406 A1 an injection waveform for performing a threshold testing in an implantable medical device is generated such that tests may be performed during the implant procedure or during a device checking procedure. The threshold test may include induction of an arrhythmia, such as ventricular fibrillation, followed by the delivery of therapy at various progressively increasing stimulation parameters to terminate the arrhythmia. The induction of the arrhythmia may be accomplished via a delivery of a relatively low energy shock or through the delivery of an induction stimulation pulse to the cardiac tissue timed concurrently with the vulnerable phase of the cardiac cycle.

U.S. Publication No. 2003/0195569 A1 discloses a method for determining a cardiac shock strength, for example a programmed first therapeutic shock strength of an implantable cardioverter defibrillator, including sensing a change in a T wave of an electrogram with respect to time, delivering a test shock by delivering a test shock at a test shock strength and at a test shock time relating to a maximum of a first derivative of the T wave with respect to time, and sensing for cardiac fibrillation. The implantable cardioverter defibrillator herein is designed for a transvenous implantation.

Another transvenous cardioverter defibrillator device is disclosed, for example, in U.S. Pat. No. 7,386,342.

The present disclosure is directed toward overcoming one or more of the above-mentioned problems, though not necessarily limited to embodiments that do.

### SUMMARY

It is an object of the instant invention to provide a non-transvenous implantable cardioverter defibrillator device and a method for operating a non-transvenous implantable cardioverter defibrillator device which allow for a threshold testing with a minimum impact on a patient when causing a fibrillation state of a patient's heart.

In one aspect, a non-transvenous implantable cardioverter defibrillator device comprises a generator device comprising a processing circuitry and a shock generation circuitry, and at least one lead comprising a shock electrode for emitting electrical shock pulses externally to a patient's heart. Said processing circuitry is configured to identify a sensed ventricular contraction event in a sensed electrocardiogram signal, or said processing circuitry is configured to control said shock generation circuitry to generate at least one conditioning pulse for emission by said shock electrode to cause an induced ventricular contraction event. Said processing circuitry is further configured to control said shock generation circuitry to generate a fibrillation pulse for emission by said shock electrode at a delay time after said sensed or induced ventricular contraction event in order to induce a cardiac fibrillation state. A non-transvenous implantable cardioverter defibrillator device (in short non-transvenous ICD) generally is configured to emit a shock pulse for achieving a defibrillation. The non-transvenous implantable cardioverter defibrillator device may serve for monitoring and treating potentially life-threatening arrhythmias of a patient's heart. The non-transvenous implantable cardioverter defibrillator device is configured for non-transvenous implantation, that is an implantation such that no electrode leads transvenously are implanted within the heart of a (human or animal) patient. The non-transvenous implantable cardioverter defibrillator device hence is to be implanted in a patient such that a generator and an electrode arrangement, for example a shock electrode placed on a lead connected to the generator, are implanted extracardially and do not reach into the heart of the patient, that is into the right or left ventricle or the right or left atrium.

The non-transvenous implantable cardioverter defibrillator device comprises a lead to be implanted extracardially and a shock electrode arranged on the lead. The shock electrode herein, in an implanted state of the defibrillator device, is placed outside of the heart of the patient, for example in the region of the sternum of the patient, such that a shock pulse for achieving a defibrillation is generated outside of the heart. Generally, the non-transvenous implantable cardioverter defibrillator device does not comprise any portions which extend transvenously into the heart, but the defibrillator device is configured to achieve a sensing and emission of signals outside of the heart.

Within the non-transvenous implantable cardioverter defibrillator device the processing circuitry is designed to evaluate information about ventricular activity. In particular, the processing circuitry, in one embodiment, may be configured to identify a sensed ventricular contraction event in a sensed electrocardiogram signal. The processing circuitry hence is configured to identify a ventricular contraction event stemming from intrinsic cardiac activity.

In another embodiment, the processing circuitry is configured to control the shock generation circuitry to generate one or multiple conditioning pulses for emission by the shock electrode in order to cause an induced ventricular contraction event. The processing circuitry hence induces a ventricular contraction event by causing the shock generation circuitry to generate and emit, using the shock electrode, one conditioning pulse or a sequence of conditioning pulses, such that a cardiac activity is forced.

Subsequent to the ventricular contraction event, may it be sensed or induced, the processing circuitry is configured to control the shock generation circuitry to generate a fibrillation pulse for emission by the shock electrode. The fibrillation pulse herein is emitted at a delay time after the sensed or induced ventricular contraction event in order to induce a cardiac fibrillation state. By means of the fibrillation pulse, hence, the patient's heart is forced to transition from a normal state to a fibrillation state in which the heart exhibits fibrillation.

The delay time is set such that the fibrillation pulse subsequent to the sensed or induced ventricular contraction event preferably coincides with the vulnerable phase of the rising T wave subsequent to the prior ventricular contraction. In that the fibrillation pulse falls into the vulnerable phase, the fibrillation pulse effectively and at a low pulse energy may cause the patient's heart to transition into the fibrillation state.

The processing circuitry may be configured to control the shock generation circuitry to generate a fibrillation pulse for emission by said shock electrode at a delay time after a sensed ventricular contraction event, or may be configured to control the shock generation circuitry to generate a fibrillation pulse for emission by said shock electrode at a delay time after an induced ventricular contraction event. The processing circuitry in one embodiment may implement one or the other control scheme. In another embodiment, the processing circuitry may implement both control schemes and hence may be enabled to control the shock generation circuitry to generate a fibrillation pulse based on either a sensed ventricular contraction event or an induced ventricular contraction event.

In that the fibrillation pulse is timed with respect to a prior ventricular contraction event and is emitted at a specific delay time subsequent to the prior ventricular contraction event to coincide with the vulnerable phase of the subsequent T wave, a low energy pulse may be sufficient to cause the fibrillation state. In that way, the impact and stress on the patient for causing the transition to the fibrillation state is reduced to a minimum. In that the emission of the fibrillation pulse is timed with respect to a prior ventricular contraction event, an easy and reliable processing may be achieved, which does not rely on the detection of a current T wave for emitting the fibrillation pulse.

Subsequent to entering the fibrillation state, a threshold testing may be performed. Generally, during the threshold testing a positioning of the lead with the shock electrode and potentially other electrode poles arranged thereon may be tested. In addition, a general setup of the non-transvenous implantable cardioverter defibrillator device may be adapted in order to achieve an effective defibrillation during subsequent operation of the non-transvenous implantable cardioverter defibrillator device. During the threshold testing, for example, the device's capability of detecting and terminating an arrhythmia may be assessed.

In that the fibrillation pulse to cause a transition to the fibrillation state is timed with respect to a sensed or induced ventricular contraction event, no detection of a T wave at the time of emission of the fibrillation pulse is required. Generally, the delay time is preprogrammed or predefined according to a prior sensing of cardiac activity such that the emission of the fibrillation pulse may reliably be caused subsequent to a sensed ventricular contraction event or subsequent to inducing a ventricular contraction event.

A ventricular contraction event is identified in an electrocardiogram signal according to the QRS complex. A timing of a ventricular contraction event may in particular be identified based on the R peak in the QRS complex. Subsequent to the R peak, namely at a timing distance corresponding to the delay time subsequent to the R peak, the fibrillation pulse is emitted, the delay time being set such that the fibrillation pulse falls into the cardiac vulnerable phase in order to cause a fibrillation state of the patient's heart.

In one embodiment the non-transvenous implantable cardioverter defibrillator device comprises a sensing arrangement for sensing electrocardiogram signals. Sensed signals are forwarded to the processing circuitry, which processes the signals in order to identify a ventricular contraction event in a sensed electrocardiogram signal. Based on a sensed ventricular contraction event, then, a fibrillation pulse may be generated in order to cause a transitioning into the fibrillation state.

The sensing arrangement may comprise multiple electrode poles. One or multiple electrode poles of the sensing arrangement herein may be placed on the lead carrying the shock electrode. For example, one electrode pole may be placed on the lead at a position proximal to the shock electrode. Another electrode pole may be placed on the lead at a position distal to the shock electrode.

Further electrode poles may be placed on further leads connected to the generator device. Alternatively or in addition, one or multiple electrode poles may be formed by a housing of the generator device. Yet alternatively or in addition, the shock electrode may be used as a sense electrode pole for sensing electrocardiogram signals.

In one embodiment, the sensing arrangement comprises three or more electrode poles. The three or more electrode poles form multiple pairs of electrode poles which may be used for sensing electrocardiogram signals. The different pairs span sense vectors which, each by itself, may be used to sense an electrocardiogram signal. The different sense vectors herein may exhibit a different spatial sensitivity with respect to electrocardiogram signals and hence may be used to sense information in a multichannel processing. Signals received by means of the different sense vectors as spanned by different pairs of electrode poles may be combined in order to sense ventricular activity and to derive information from electrocardiogram signals.

In one embodiment, the processing circuitry is configured to identify at least two subsequent ventricular contraction events in an electrocardiogram signal to determine a first interval based on the at least two subsequent ventricular contraction events. Ventricular contraction events may be sensed in electrocardiogram signals of one or of multiple signal vectors as spanned by one or multiple pairs of electrode poles. Information from multiple signals received by multiple pairs of electrode poles spanning different sense vectors may in particular be combined in order to reliably detect ventricular contraction events in electrocardiogram signals.

A timing of a ventricular contraction event herein may in particular be determined according to the timing of an R peak of a QRS complex. The first interval hence corresponds to the RR interval, that is the interval between subsequent R peaks of QRS complexes.

The first interval may be set according to the interval as measured between two subsequent ventricular contraction events, for example two subsequent R peaks in the electrocardiogram signal. In another embodiment, the first interval may be set according to an averaging or median of RR intervals as measured for a multiplicity of R peaks over a multiplicity of cardiac cycles.

In one embodiment, the processing circuitry is configured to set the delay time based on the first interval. In particular, the processing circuitry may set the delay time to correspond to a value in a range between 10% and 50% of the first interval.

In one embodiment, the processing circuitry is configured to identify a ventricular contraction event and a subsequent T wave in an electrocardiogram signal, to determine a second interval based on the ventricular contraction event and the subsequent T wave, and to set the delay time based on the second interval. The ventricular contraction event and the subsequent T wave may, for example, be sensed using the same sense vector of a pair of electrode poles, or using different sense vectors of different pairs of electrode poles. Information from multiple signals received using multiple pairs of electrode poles spanning different sense vectors may in particular be combined in order to reliably detect ventricular contraction events.

A timing of a ventricular contraction event herein may in particular be determined as the timing of an R peak. The timing of the T wave may in particular be determined according to the start of the T wave, that is according to the rising flank of the T wave, for example according to a threshold crossing. The second interval hence corresponds to the RT interval, that is the interval between an R peak and a subsequent start of a T wave.

In one embodiment, the second interval may be set according to an average or median of RT intervals as measured for a multiplicity of cardiac cycles.

In one embodiment, the processing circuitry is configured to set the delay time to correspond to a value in a range between 50% and 120% of the second interval. The delay time, at which the fibrillation pulse is to be emitted subsequent to a prior ventricular contraction event, may in particular be timed such that it may occur shortly before the start of a T wave subsequent to a ventricular contraction event or during a rising flank of the T wave subsequent to a prior ventricular contraction event.

The processing circuitry may be configured to set the delay time, for example in an initial calibration phase prior to emitting the fibrillation pulse. In another embodiment, the delay time may be fixedly set, for example preprogrammed by a user.

In yet another embodiment, information relating to RR intervals and RT intervals may be forwarded by the non-transvenous implantable cardioverter defibrillator device to an external device, which may visualize RR intervals and RT intervals to a user. The external device may comprise circuitry for automatically determining the delay time according to an automatic assessment in a simulator. Alternatively, the external device may allow a user to program the delay time based on a visualization of RR intervals and RT intervals. The external device may also allow a user to program sense vectors for sensing RR or RT intervals. The delay time as determined by the external device or as programmed by a user using the external device may be transmitted to the non-transvenous implantable cardioverter defibrillator device, which in operation may use the delay time in order to generate and emit a fibrillation pulse to cause a fibrillation state of a patient's heart.

In one embodiment, the processing circuitry is configured to control the shock generation circuitry to generate two or more conditioning pulses for emission by the shock electrode. By means of the conditioning pulses an induced ventricular contraction event is caused, subsequent to which the fibrillation pulse is emitted to force the patient's heart to enter into a fibrillation state.

By means of the conditioning pulses the patient's heart shall be paced such that ventricular contraction events occur according to the conditioning pulses. The conditioning pulses hence are designed such that cardiac activity is caused by the conditioning pulses. By means of the conditioning pulses it can be made sure that, at least at the last conditioning pulse, a ventricular contraction event occurs. Subsequent to the last conditioning pulse the fibrillation pulse is emitted, the fibrillation pulse having a time distance corresponding to the delay time with respect to the last fibrillation pulse.

In case conditioning pulses are used to force an (induced) ventricular contraction event, the delay time may be set according to the conditioning interval. For example, the delay time may be set by the processing circuitry to correspond to a value in a range between 50% and 120% of the conditioning interval.

The conditioning pulses are spaced apart by a time distance corresponding to the conditioning interval, which may be significantly shorter than the first interval (corresponding to the intrinsic RR interval). For example, the conditioning interval may lie in a range between 10% and 60% of the first interval, the first interval being indicative of the intrinsic RR interval and hence to an intrinsic cardiac cycle interval time.

The fibrillation pulse may exhibit a single phase. The fibrillation pulse may, in another embodiment, exhibit multiple phases. Within the multiple phases polarities of the pulse may change.

In one embodiment, the shock generation circuitry comprises a multiplicity of energy storage devices, for example in the shape of capacitors, and at least one switching device. The processing circuitry herein is configured to control the at least one switching device to supply energy for generating the fibrillation pulse using all of the multiplicity of energy storage devices or a combination of some of the multiplicity of energy storage devices. The fibrillation pulse in particular may be produced by causing energy storage devices in the shape of capacitors to discharge.

The processing circuitry herein may be controlled to produce a fibrillation pulse which is shaped as a rectangular or approximately rectangular pulse. Alternatively, the fibrillation pulse may be shaped to exhibit a rising ramp.

The fibrillation pulse may have a duration between 0.5 to 50 ms.

The fibrillation pulse may have an energy in between 1 to 50 J.

The fibrillation pulse may have a maximum voltage in a range between 5 to 800 V.

The processing circuitry may be designed to control the shock generation circuitry to generate one or multiple conditioning pulses at a level smaller than the fibrillation pulse. For example, the conditioning pulses may exhibit a voltage level at or smaller than 80% of the maximum voltage level of the fibrillation pulse.

In one embodiment, the processing circuitry is configured to control the at least one switching device to supply energy for generating the fibrillation pulse using a first combination of the multiplicity of energy storage devices and to supply energy for generating the at least one conditioning pulse using a second combination of the multiplicity of energy storage devices different than the first combination. For example, for producing the fibrillation pulse all of the energy storage devices, for example in the shape of capacitors, may be used. For producing the conditioning pulses, in contrast, only a subgroup of the energy storage devices, for example in the shape of capacitors, may be used, such that the conditioning pulses are produced at a reduced level and at a reduced energy in comparison to the fibrillation pulse.

In one embodiment, the processing circuitry is configured to control the shock generation circuitry to generate the at least one conditioning pulse using a pulse width modulation. The conditioning pulses hence each are produced by a burst of short pulses according to a pulse width modulation scheme.

Within the non-transvenous implantable cardioverter defibrillator device, subsequent to emitting a fibrillation pulse, it may be assessed whether the patient's heart has entered into a fibrillation state. If a fibrillation state is detected, for example a special testing may be performed. If it is found that the patient's heart has not entered into a fibrillation state, another fibrillation pulse may be emitted, wherein parameters for the next fibrillation pulse may be adapted, for example by increasing its voltage level and/or its energy. The adaption of the fibrillation pulse may be performed automatically by the system. Alternatively, a user may be prompted to program or confirm the parameters for the fibrillation pulse, wherein the system may propose changes for confirmation by the user. The emission of fibrillation pulses may be repeated until a fibrillation state is successfully detected. A user may be enabled to terminate the emission of the fibrillation pulses.

The generator device may have a volume smaller than 70 ccm.

The non-transvenous implantable cardioverter defibrillator device may be MRI compatible.

The non-transvenous implantable cardioverter defibrillator device may comprise a communication interface for communicating with an external device, for example within a home-monitoring system. The communication interface may, for example, employ a common communication scheme such as a MICS communication or a BLE communication.

In another aspect, in a method for operating a non-transvenous implantable cardioverter defibrillator device the non-transvenous implantable cardioverter defibrillator device comprises a generator device and at least one lead having a shock electrode for emitting electrical shock pulses externally to a patient's heart. The method comprises: identifying, using a processing circuitry of the generator device, a sensed ventricular contraction event in a sensed electrocardiogram signal or controlling, using said processing circuitry of the generator device, a shock generation circuitry of the generator device to generate at least one conditioning pulse for emission by said shock electrode to cause an induced ventricular contraction event; and controlling, using said processing circuitry, said shock generation circuitry to generate a fibrillation pulse for emission by said shock electrode at a delay time after said sensed or induced ventricular contraction event in order to induce a cardiac fibrillation state.

The advantages and advantageous embodiments described above for the non-transvenous implantable cardioverter defibrillator device equally apply also to the method, such that it shall be referred to the above in this respect.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The idea of the present invention shall subsequently be described in more detail with reference to the embodiments as shown in the drawings. Herein:

FIG. 1 shows a schematic drawing of a non-transvenous implantable cardioverter defibrillator device in an implanted state in a patient;

FIG. 2 shows the non-transvenous cardioverter defibrillator device of FIG. 1, illustrating sense vectors spanned by different pairs of electrode poles of a sensing arrangement of the device:

FIG. 3 shows a schematic drawing of an electrocardiogram signal:

FIG. 4 shows a schematic drawing of a fibrillation pulse in relation to an electrocardiogram signal:

FIG. 5 shows conditioning pulses and a fibrillation pulse in relation to an electrocardiogram signal;

FIG. 6 shows an example of a shock generation circuitry for generating a fibrillation pulse; and

FIG. 7 shows another example of a shock generation circuitry for generating a fibrillation pulse.

### DETAILED DESCRIPTION

Subsequently, embodiments of the present invention shall be described in detail with reference to the drawings. In the drawings, like reference numerals designate like structural elements.

It is to be noted that the embodiments are not limiting for the present invention, but merely represent illustrative examples.

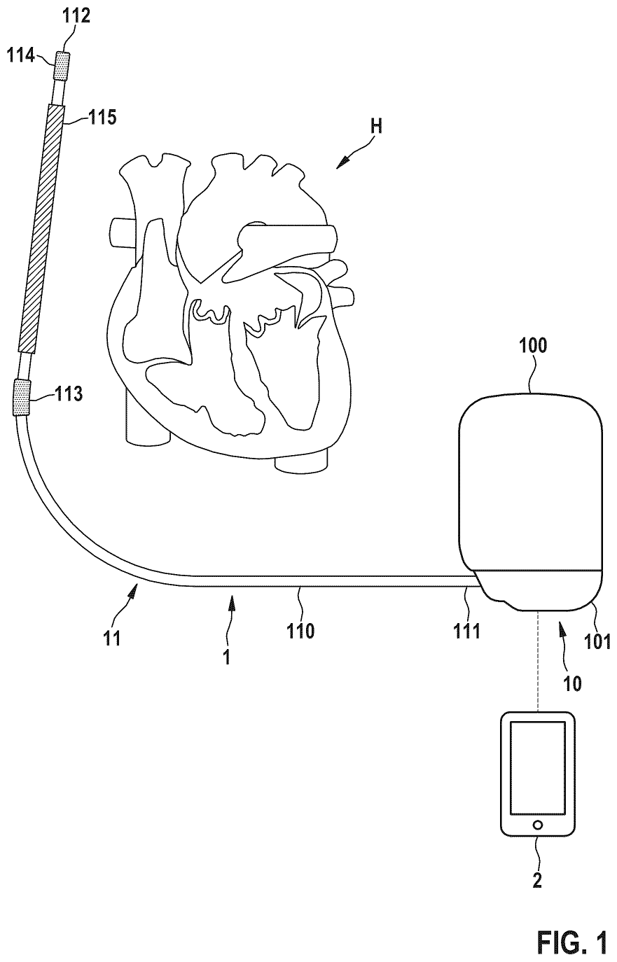

Referring to FIG. 1, in a setup of a therapy system a non-transvenous implantable cardioverter defibrillator device 1 is implanted such that the non-transvenous implantable cardioverter defibrillator device 1 is completely external to the heart H, the non-transvenous implantable cardioverter defibrillator device 1 comprising a generator device 10 encapsulated within a housing 100, and a lead 11 connected to the generator device 10 at a proximal end 111 and carrying electrode poles 113, 114 as well as a shock electrode 115 in the shape of a coil formed on a distal portion close to a distal end 112 of the lead 11. The electrode poles 113, 114, for example formed as ring electrodes on either side of the shock electrode 115, serve to sense cardiac signals for processing within the generator device 10 of the non-transvenous implantable cardioverter defibrillator device 1, such that based on sensed signals an arrhythmia may be identified and a shock pulse may be generated for providing for a defibrillation therapy.

The non-transvenous implantable cardioverter defibrillator device 1 is designed for a non-transvenous implantation, that is an implantation external to the patient's heart H. In particular, the lead 11 connected to the generator device 10 shall rest outside of the patient's heart H and shall not extend transvenously into the heart, the shock electrode 115 hence, in an implanted state, being placed outside of the heart H for providing for a defibrillation therapy.

For example, the generator device 10 may be implanted subcutaneously in a patient. The lead 11, with a lead body 110, may extend from the generator device 10 towards the sternum of the patient, the lead 11, for example, tunneling through tissue in the region of the sternum and being placed beneath the sternum of the patient.

The non-transvenous implantable cardioverter defibrillator device 1 may comprise a communication interface for communicating with an external device 2, for example within a home-monitoring system.

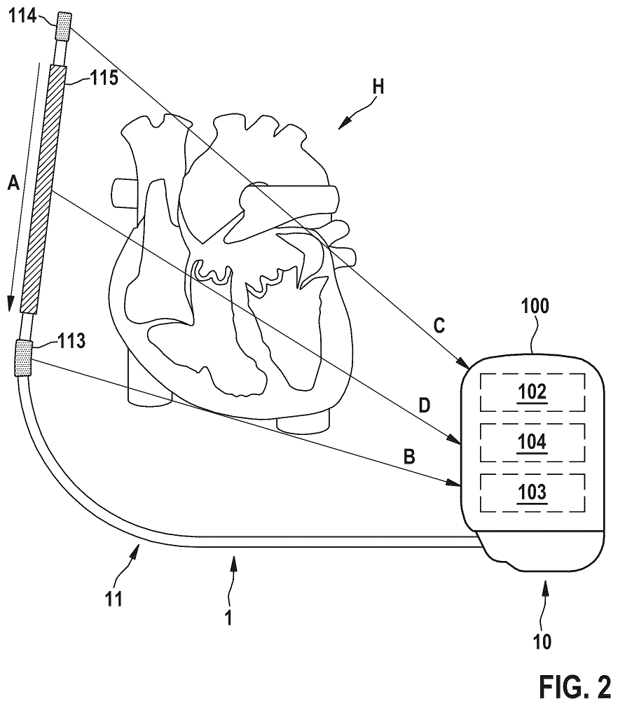

Referring now to FIG. 2, the generator device 10 generally comprises a processing circuitry 102 for controlling operation of the non-transvenous implantable cardioverter defibrillator device 1. In addition, the generator device 10 comprises a shock generation circuitry 103 and an energy storage 104, in particular in the shape of a battery.

The processing circuitry 102 in particular serves to process signals sensed via a sensing arrangement formed by the electrode poles 113, 114 arranged on the lead 11 and additional poles, such as the shock electrode 115 and the housing 100 of the generator device 10. The different poles of the sensing arrangement form pairs of electrode poles in between which sense vectors A, B, C, D are spanned, as illustrated in FIG. 2, the different sense vectors A, B, C, D allowing to sense electrocardiogram signals from the patient's heart H with a different spatial sensitivity, hence allowing to sense and process information from the patient's heart H in a multichannel processing.

The non-transvenous implantable cardioverter defibrillator device 1 as shown in FIGS. 1 and 2 in particular shall be configured to perform a defibrillation therapy. Herein, upon implantation of the non-transvenous implantable cardioverter defibrillator device 1 it shall be assessed whether the non-transvenous implantable cardioverter defibrillator device 1 is enabled for operation by employing a so-called threshold testing. Within the threshold testing it in particular is checked whether, using the non-transvenous implantable cardioverter defibrillator device 1, an arrhythmia may reliably be detected, and a defibrillation therapy may be performed in order to overcome the arrhythmia.

For performing the threshold testing, the patient's heart H shall be set into a fibrillation state in which the patient's heart H exhibits fibrillation. Once the patient's heart H is in the fibrillation state, it may be assessed whether the arrhythmia may be detected, and a therapy by delivering a defibrillation shock using the shock electrode 115 may be provided in order to end the arrhythmia.

For causing a transition of the patient's heart H into the fibrillation state, in an initial phase prior to actual operation, or in a recalibration phase between periods of operation, the processing circuitry 102 shall cause the shock generation circuitry 103 to emit a fibrillation pulse using the shock electrode 115, the fibrillation pulse being designed to cause the patient's heart H to enter into the fibrillation state.

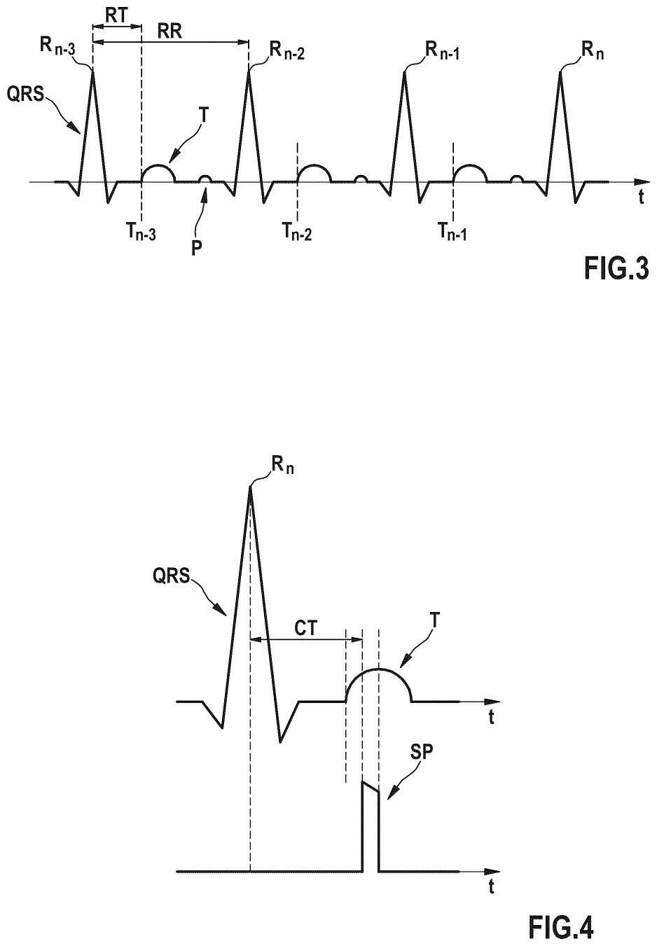

Referring now to FIG. 3, using one or multiple pairs of electrode poles as illustrated in FIG. 2 electrocardiogram signals may be sensed and processed using the processing circuitry 102. Electrocardiogram signals in general exhibit a shape similarly as illustrated in FIG. 3, ventricular contraction events being discernible within the electrocardiogram signal according to a so-called QRS complex, which in a cardiac cycle is followed by a so-called T wave and is preceded by a so-called P wave. Whereas the QRS complex represents a ventricular contraction, the T wave corresponds to the depolarization of the ventricles following ventricular contraction. The P wave in turn represents atrial activity preceding a ventricular contraction.

Employing one or multiple sense vectors A, B, C, D of one or multiple different pairs of electrode poles, events in an electrocardiogram signal may be sensed and identified using the processing circuitry 102 of the generator device 10.

In particular, the processing circuitry 102 may be configured to determine R peaks Rₙ₋₃ . . . Rₙ of ventricular contraction events. Based on the R peaks Rₙ₋₃ . . . Rₙ a first interval corresponding to the so-called RR interval may be determined based on a time distance between two subsequent R peaks Rₙ₋₃ . . . Rₙ. For setting the first interval, an average or a median over multiple cardiac cycles may be formed.

In addition, the processing circuitry 102 may be configured to determine the start of the T wave Tₙ₋₃ . . . Tₙ₋₁ in cardiac cycles. In particular, the start of a T wave may be determined based on a rising flank of the T wave, for example based on a threshold crossing of a signal relating to the T wave. Based on an identified T wave a second interval RT may be determined based on a time distance between an R peak Rₙ₋₃ . . . Rₙ and a subsequent T wave Tₙ₋₃ . . . Tₙ₋₁. For setting the second interval, an average or a median over multiple cardiac cycles may be formed.

Referring now to FIG. 4, in one embodiment the processing circuitry 102 is configured to control the shock generation circuitry 103 to emit a fibrillation pulse in order to cause a transition of the patient's heart H into a fibrillation state. The processing circuitry 102 herein senses in an electrocardiogram signal an R peak Rₙ relating to a ventricular contraction event, and causes the shock generation circuitry 103 to generate a fibrillation pulse SP for emission by the shock electrode 115 at a delay time CT subsequent to the R peak Rₙ of the ventricular contraction event. Hence, based on a sensed ventricular activity a fibrillation pulse SP is generated and emitted with a timing with respect to the prior ventricular contraction such that the fibrillation pulse SP coincides with a beginning phase of the T wave, which corresponds to a vulnerable phase in which a low energy pulse may be sufficient in order to cause the patient's heart H to enter into fibrillation.

The delay time CT may be preprogrammed and hence may be fixedly set.

Alternatively, the delay time CT may be set by the processing circuitry 102 according to the first interval RR or the second interval RT as determined over one or multiple cardiac cycles in a prior phase of operation. For example, the delay time CT may be set to a value in a range between 50% and 120% of the second interval RT.

In yet another embodiment, the generator device 10 may forward information relating to RR intervals and RT intervals to an external device 2 (see FIG. 1), which may conduct an automatic evaluation or which may visualize electrocardiogram information to a user, which may program the delay time CT according to the information provided via the external device 2.

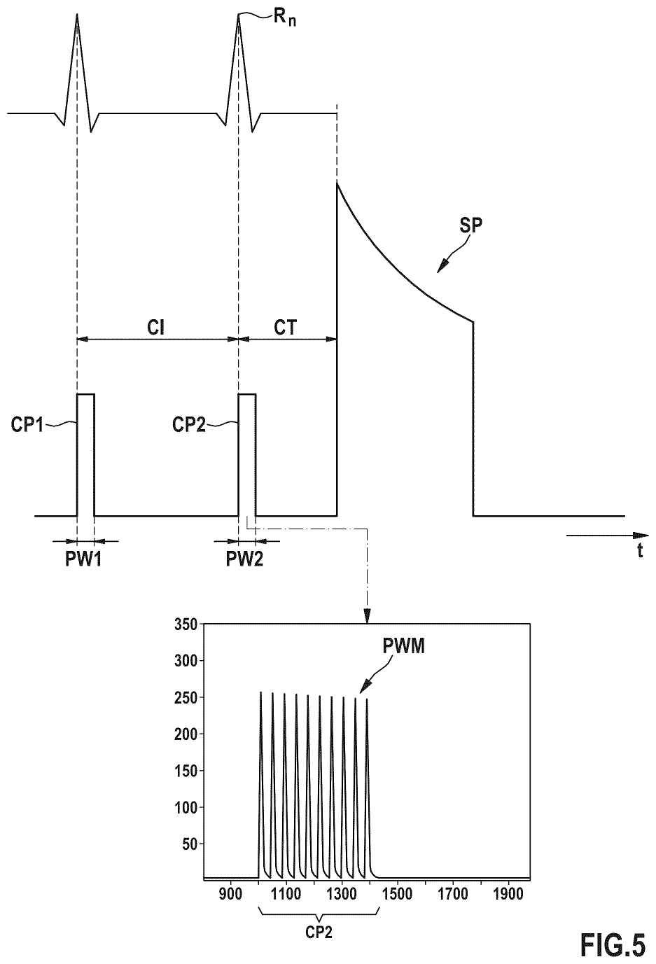

Referring now to FIG. 5, in one embodiment the processing circuitry 102 is configured to control the shock generation circuitry 103 to emit conditioning pulses CP1, CP2 prior to emitting a fibrillation pulse SP. The conditioning pulses CP1, CP2, having pulse widths PW1, PW2 which may be equal or different from one another, shall induce ventricular contractions of the patient's heart such that the heart rhythm is temporarily forced to follow the conditioning pulses CP1, CP2. Subsequent to the last conditioning pulse CP2, which (presumably) coincides with an induced ventricular contraction event Rₙ, the processing circuitry 102 causes the shock generation circuitry 103 to generate a fibrillation pulse SP, the fibrillation pulse SP causing the patient's heart H to enter into fibrillation.

The conditioning pulses CP1, CP2, in the shown embodiment, are spaced apart by a time distance corresponding to a conditioning interval C1. The conditioning interval C1 may, for example, be set according to the first interval RR. For example, the conditioning interval C1 may be set to a value in a range in between 10% to 60% of the first interval, such that the conditioning pulses CP1, CP2 are emitted at a rate significantly faster than the intrinsic heart rate.

The fibrillation pulse SP is emitted at a time distance corresponding to the delay time CT subsequent to the last conditioning pulse CP2. The delay time CT may be set according to the conditioning interval C1, for example to a value in a range between 50% to 120% of the conditioning interval C1.

As illustrated in FIG. 5, the conditioning pulses CP1, CP2 may be formed by employing a pulse width modulation PWM. Within the pulse width modulation one or both conditioning pulses CP1, CP2 may be formed by a sequence of pulse bursts which together make up the respective conditioning pulse CP1, CP2.

The processing circuitry 102 may implement either the control scheme according to FIG. 4 or the control scheme of FIG. 5. In another embodiment, the processing circuitry 102 may implement both control schemes, wherein one control scheme or the other may be selectively used to cause an emission of a fibrillation pulse to cause the patient's heart H to enter into fibrillation.

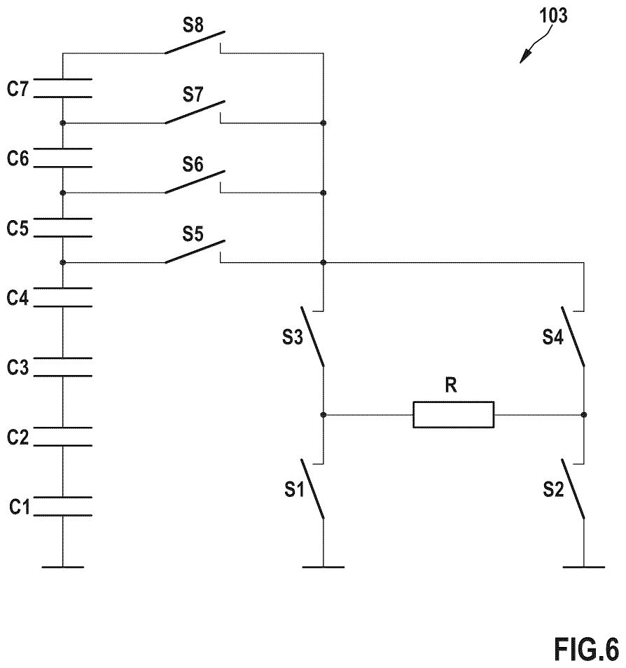

Referring now to FIG. 6, the shock generation circuitry 103 may be implemented by an arrangement of energy storage devices formed by capacitors C1 to C7 and an arrangement of switching devices S1 to S8. The capacitors C to C7 are in operative electrical connection to the energy storage 104, formed by a battery, of the generator device 10 and may be charged by the energy storage 104 in order to generate electrical pulses for emission by the shock electrode 115.

In the embodiment of FIG. 6, the capacitors C1 to C7 are connected to each other in an electrical series connection. Switching devices S5 to S8 selectively connect the capacitors C1 to C7 to a so-called H bridge formed by switching devices S1 to S4. R represents an effective body impedance, the switching devices S1 to S4 selectively forming therapeutic current paths for emitting electrical pulses of a desired polarity into the patient's body.

By means of the switching devices S5 to S8 the electrical voltage of an electrical output pulse may be set. If only the switching device S5 is closed, the electrical pulse is formed by the charge of the capacitors C1 to C4, which discharge via the electrical path formed by the closed switching device S5. The electrical pulse is fed through the H bridge, wherein either the combination of switching devices S3, S2 or the combination of switching devices S4, S1 is closed in order to form an electrical pulse at a particular polarity for emission into the body of the patient.

In order to set the voltage level of the electrical pulse, either one of the switching devices S5 to S8 is closed. If the switching device S6 instead of the switching device S5 is closed, the electrical pulse is formed by discharging the combination of the capacitors C1 to C5. If instead the switching device S7 is closed, the charge of the capacitor C6 is added. If the switching device S8 is closed, the electrical pulse is formed by the combination of all capacitors C1 to C7.

By combining all capacitors C1 to C7 by closing (only) the switching device S8, a maximum voltage for the electrical pulse may be set.

In one embodiment, the fibrillation pulse is formed by the combination of all capacitors C1 to C7 by closing the switching device S8 (and leaving the switching devices S5 to S7 open). For forming the fibrillation pulse, herein, the capacitors C1 to C7 are charged by the energy storage 104 of the generator device 10 to such a level that the voltage of the fibrillation pulse is set to a desired level.

Conditioning pulses instead may be formed by using a subgroup of the capacitors C1 to C7. For example, the conditioning pulses may be formed by closing (only) the switching device S5 in order to form the conditioning pulses by discharging the capacitors C to C4. The conditioning pulses hence exhibit a reduced voltage level in comparison to the fibrillation pulse.

The conditioning pulses may, in one embodiment, be formed by employing a pulse width modulation technique. For this, for example the switching device S5 may be repeatedly opened and closed (while the switching devices S6 to S8 are in the open position), such that pulse bursts are formed, as illustrated in FIG. 5.

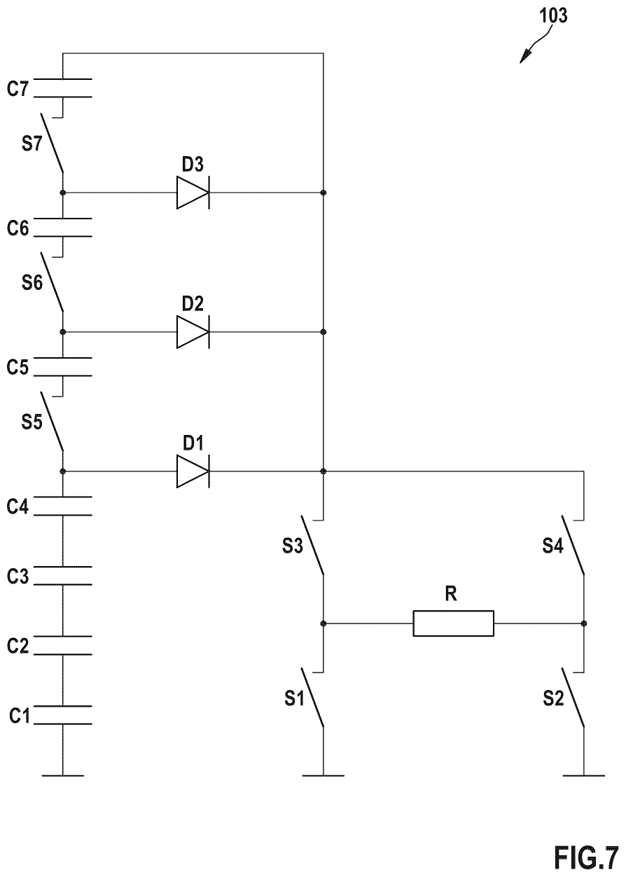

Referring now to FIG. 7, in another embodiment the shock generation circuitry 103 comprises energy storage devices in the shape of capacitors C1 to C7, which each may be charged with electrical energy supplied from the energy storage 104 (FIG. 2) in the shape of a battery. The shock generation circuitry 103 in addition comprises switching devices S5-S7, which serve to selectively couple the energy storage devices C1 to C7 to an output circuitry in the shape of an H bridge comprising switching devices S1 to S4 for selectively forming a therapeutic current path via an associated diode D1-D3 for injecting a shock pulse into a patient, represented in the schematic circuit diagram of FIG. 7 by an effective body impedance R.

In the embodiment of FIG. 7, electrical pulses may be formed by selectively switching the switching devices S5 to S7 to a closed position. Herein, if all switching devices S5 to S7 are open, an electrical pulse is formed by the combination of the capacitors C1 to C4. By closing the switching device S5, the capacitor C5 is added to the combination, such that an electrical pulse is formed by discharging the combination of the capacitors C1 to C5. By closing also the switching device S6, the capacitor C6 is added. By closing all switching devices S5 to S7, an electrical pulse of a maximum voltage level is produced from the combination of all capacitors C1 to C7 along the electrical conduction path via diode D3 towards the H bridge formed by the switching devices S1 to S4.

By combining all capacitors C1 to C7 by closing all switching devices as S5 to S7, hence, a maximum voltage for the electrical pulse may be set. As described above, the fibrillation pulse may be formed by the combination of all capacitors C1 to C7 by closing the switching devices S5 to S7. The capacitors C1 to C7 may be charged by the energy storage 104 of the generator device 10 to such a level that the voltage of the fibrillation pulse is set to a desired level.

As described above in connection to FIG. 6, conditioning pulses instead may be formed by using a subgroup of the capacitors C1 to C7. For example, the conditioning pulses may be formed by keeping all switching devices S5 to S7 in the open state in order to form the conditioning pulses by discharging the capacitors C to C4. The conditioning pulses hence exhibit a reduced voltage level in comparison to the fibrillation pulse.

The conditioning pulses may be formed by employing a pulse width modulation technique. For this, the switching devices S1 to S4 of the H bridge may be used. For example, if the H bridge is switched to form an electrical conduction path via switching devices S3, S2 to generate a pulse of a desired polarity, the switching device S3 may repeatedly be opened in order to open the conduction path for achieving a pulse width modulation.

The circuit arrangements of FIGS. 6 and 7 allow to shape an output pulse, for example a fibrillation pulse, such that it may exhibit an approximately rectangular pulse waveform or another desired waveform.

In particular, for generating an output pulse, e.g., in the embodiment of FIG. 7 the switching devices S5 to S7 in a first time span may be in an open state, such that the arrangement of energy storage devices C1 to C4 are connected to the H bridge, the energy storage devices C1 to C4 being connected in series. During the first time span the energy storage devices C1 to C4 discharge via the diode D1 for supplying energy to the H bridge.

In a subsequent, second time span, the switching device S5 is closed, such that the energy storage device C5 is connected in series to the energy storage devices C1 to C4, such that energy now is supplied to the H bridge via the diode D2 (due to the voltage being supplied from the energy storage device C5, the diode D1 assumes a blocking state, the diode D2 in turn assuming a conducting state such that the energy is supplied via the diode D2 to the H bridge circuit).

In a third time span the switching device S6 is closed (while the switching device S5 remains closed), such that the further energy storage device C6 is connected in series to the energy storage devices C1 to C5, and energy is supplied to the H bridge via diode D3 (when the switching devices S5, S6 are closed, the diodes D1, D2 are in a blocking state, such that energy is supplied via the diode D3 to the H bridge).

Further energy storage devices C7 beyond the energy storage devices C1 to C6 may be added, which are connected each via an associated switching device S7 in series to the energy devices C1 to C6 below, as illustrated in FIG. 7.

By consecutively adding energy storage devices C1 to C7 for the shaping of the output pulse, the output pulse may assume a waveform which substantially resembles a rectangular waveform, or another waveform, for example a rising ramp or the like.

The idea underlying the present invention is not limited to the embodiments described above, but may be implemented in an entirely different fashion.

A non-transvenous implantable cardioverter defibrillator device may comprise one or multiple leads, with one or multiple electrode poles arranged on each lead. The leads are configured for implantation external to the patient's heart, such that no portions of the non-transvenous implantable cardioverter defibrillator device extend transvenously into the heart.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Non-transvenous implantable cardioverter defibrillator device
10 Generator device
100 Housing
101 Connection block
102 Processing circuitry
103 Shock generation circuitry
104 Energy storage (battery)
11 Electrode lead
110 Lead body
111 Proximal end
112 Distal end
113, 114 Electrode pole
115 Shock electrode (coil)
2 External device
A-D Reception vector
C1-C7 Capacitors
C1 Conditioning interval
CP1, CP2 Conditioning pulse
CT Delay time
D1-D3 Diode
H Heart
P wave P
PW1, PW2 Pulse width
PWM Pulse-width modulated pulses
QRS QRS complex
R Resistance
Rₙ₋₃ . . . Rₙ R peak
RR Cardiac interval (RR interval)
RT RT interval
S1-S8 Switches
SP Fibrillation shock pulse
T T wave

1. A non-transvenous implantable cardioverter defibrillator device comprising
a generator device comprising a processing circuitry and a shock generation circuitry; and
at least one lead comprising a shock electrode for emitting electrical shock pulses externally to a patient's heart;
wherein said processing circuitry is configured to identify a sensed ventricular contraction event in a sensed electrocardiogram signal or said processing circuitry is configured to control said shock generation circuitry to generate at least one conditioning pulse for emission by said shock electrode to cause an induced ventricular contraction event;
wherein said processing circuitry is further configured to control said shock generation circuitry to generate a fibrillation pulse for emission by said shock electrode at a delay time CT after said sensed or induced ventricular contraction event in order to induce a cardiac fibrillation state.

2. The non-transvenous implantable cardioverter defibrillator device according to claim 1, wherein a sensing arrangement for sensing said electrocardiogram signal.

3. The non-transvenous implantable cardioverter defibrillator device according to claim 2, wherein at least one electrode pole of said sensing arrangement is arranged on said at least one lead.

4. The non-transvenous implantable cardioverter defibrillator device according to claim 2, wherein at least one electrode pole of said sensing arrangement is formed by a housing of said generator device.

5. The non-transvenous implantable cardioverter defibrillator device according to claim 2, wherein said sensing arrangement includes at least three electrode poles, the processing circuitry being configured to sense electrocardiogram signals using different pairs of electrode poles of said at least three electrode poles.

6. The non-transvenous implantable cardioverter defibrillator device according to claim 1, wherein the processing circuitry is configured to identify at least two subsequent ventricular contraction events in an electrocardiogram signal, to determine a first interval based on the at least two subsequent ventricular contraction events.

7. The non-transvenous implantable cardioverter defibrillator device according to claim 1, wherein the processing circuitry is configured to identify a ventricular contraction event a subsequent T wave in an electrocardiogram signal, to determine a second interval based on the ventricular contraction event and the subsequent T wave, and to set said delay time based on the second interval.

8. The non-transvenous implantable cardioverter defibrillator device according to claim 7, wherein the processing circuitry is configured to set said delay time to correspond to a value in a range between 50% and 120% of the second interval.

9. The non-transvenous implantable cardioverter defibrillator device according to the claim 1, wherein the processing circuitry is configured to control said shock generation circuitry to generate at least two conditioning pulses for emission by said shock electrode to cause said induced ventricular contraction event, said at least two conditioning pulses being spaced apart by a conditioning interval.

10. The non-transvenous implantable cardioverter defibrillator device according to claim 9, wherein the processing circuitry is configured to set said delay time to correspond to a value in a range between 50% and 120% of the conditioning interval.

11. The non-transvenous implantable cardioverter defibrillator device according to claim 9, wherein the processing circuitry is configured to set said conditioning interval based on a cardiac cycle interval time.

12. The non-transvenous implantable cardioverter defibrillator device according to claim 9, wherein the shock generation circuitry comprises a multiplicity of energy storage devices and at least one switching device, wherein the processing circuitry is configured to control the at least one switching device to supply energy for generating said fibrillation pulse using all of said multiplicity of energy storage devices or a combination of some of said multiplicity of energy storage devices.

13. The non-transvenous implantable cardioverter defibrillator device according to claim 12, wherein the processing circuitry is configured to control the at least one switching device to supply energy for generating said fibrillation pulse using a first combination of said multiplicity of energy storage devices and to supply energy for generating said at least one conditioning pulse using a second combination of said multiplicity of energy storage devices different than said first combination.

14. The non-transvenous implantable cardioverter defibrillator device according to claim 1, wherein the processing circuitry is configured to control the shock generation circuitry to generate said at least one conditioning pulse using a pulse width modulation.

15. A method for operating a non-transvenous implantable cardioverter defibrillator device, said non-transvenous implantable cardioverter defibrillator device comprising a generator device and at least one lead having a shock electrode for emitting electrical shock pulses externally to a patient's heart, the method comprising:
identifying, using a processing circuitry of the generator device, a sensed ventricular contraction event in a sensed electrocardiogram signal or controlling, using said processing circuitry of the generator device, a shock generation circuitry of the generator device to generate at least one conditioning pulse for emission by said shock electrode to cause an induced ventricular contraction event; and
controlling, using said processing circuitry, said shock generation circuitry to generate a fibrillation pulse for emission by said shock electrode at a delay time after said sensed or induced ventricular contraction event in order to induce a cardiac fibrillation state.
